(19) United States
(12) Patent Application Publication
(10) Pub. No.: US 2009/0111829 A1
(43) Pub. Date: Apr. 30, 2009

(54) Aripiprazole Salts

(75) Inventors: Laszlo Pongo, Kerepes (HU); Gyula Simig, Budapest (HU); Andras Dancso, Budapest (HU); Gyorgy Morovjan, Budapest (HU)

Correspondence Address: K.F. ROSS P.C., 5683 RIVERDALE AVENUE, SUITE 203 BOX 900, BRONX, NY, 10471-0900, US

(73) Assignee: Egis Gyógyszergyár Nyilvanosan Mukodo Reszvenytarsasag, Budapest (HU)

(21) Appl. No.: 11/988,742

(22) PCT Filed: Jul. 11, 2006
(86) PCT No.: PCT/HU2006/000056

§ 371 Date: Apr. 1, 2008

(30) Foreign Application Priority Data
Jul. 14, 2005 (HU) P0500683

(51) Int. Cl. A61K 31/496; C07D 401/12; A61P 25/18

(52) U.S. Cl. 514/253.07; 544/363

(57) ABSTRACT

The present invention provides for new salts of aripiprazole of the general formula (II) formed with dibasic organic acids, camphorsulfonic acid, phosphoric acid, and processes for their preparation. Further objects of the present invention are pharmaceutical compositions containing said new aripiprazole salts. Aripiprazole salts according to present invention can be prepared by the reaction of aripiprazole base and suitable acid compounds in a molar ratio 0.5-3 based on the molar amount of aripiprazole in a suitable organic solvent.

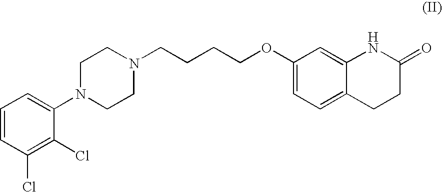

### FIELD OF THE INVENTION

The compound 7-{4-[4-(2,3-dichlorophenyl)-1-piperazinyl]butoxy}-3,4-dihydro-2(1H)-quinolinon corresponding to INN name aripiprazole of the formula

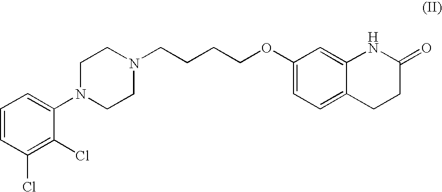

is an antipsychotic pharmaceutically active ingredient.

The present invention relates to new salts of aripiprazole formed with dibasic organic acids, camphorsulfonic acid, phosphoric acid, and processes for preparation said salts. Further objects of the present invention are pharmaceutical compositions containing said new salts of aripiprazole, furthermore the use of these salts for the treatment of psychotic diseases of the central nervous system.

Aripiprazole salts can be prepared in high purity according to the present invention with excellent properties from the point of view of pharmaceutical technology.

### TECHNICAL BACKGROUND OF THE INVENTION

Aripiprazole binds to several receptors of the central nervous system. It has high affinity to dopamine receptors D₂ and D₃, serotonine receptors 5HT_{1A} and 5HT_{2A}, binds also on dopamine receptors D₄ and serotonine receptors of 5HT_{2C} and 5HT₇, furthermore binds to the α₁-adrenerg, the hystamine H₁ receptors and the active centers of the serotonie reuptake. Aripiprazole does not bind on colinerg muscarin receptors.

Although the action mechanism of aripiprazole is not yet known except for wide-ranging different receptor bindings, its effects on psychotic diseases can be explained by agonistic interactions with dopamine D₂, serotonin 5HT_{1A} receptors and antagonistic effect to serotonin 5HT_{2A} receptors.

The advantages of use of aripiprazole for the treatment of schizophrenia and bipolar disorders are proved by clinical examinations.

7-{4-[4-(2,3-dichlorophenyl)-1-piperazinyl]butoxy}-3,4-dihydro-2(1H)-quinolinon of the formula (II) and its salts are known from the European Patent No. 367 141. Hydrate form of aripiprazole is described in the Japan patent application No. 2003212852

A new polymorph form of aripiprazole, different from the three polymorph forms known from the basic patent (EP 367 141) is described in the international patent application No. WO 2004/106322.

Pharmaceutical products have to meet large number of strict criteria of health authorities which criteria get always stricter. Moreover the evidences of adequacy of requirements have to be proved by appropriate documentations before the authorities. Said criteria concern the active pharmaceutical ingredient and also the properties of the pharmaceutical composition. These criteria are taken into consideration together during the development of pharmaceutical compositions as well as in the case of evaluation of the registration documentations by authorities.

It is known that the use of different polymorph forms of pharmaceutically active ingredients having poor solubility in water e.g. in case of use of aripiprazole base of formula (II), result different dissolution profiles of the corresponding pharmaceutical compositions, causing difficulties in the fulfillment of the requirement that the dissolution profile should be uniform even at long-term storage.

A further problem is the hydrophobic property of the pharmaceutically active ingredient in the processes for the preparation of an appropriate pharmaceutical dosage form. Therefore the active ingredients are usually converted to their salts with organic or inorganic acids and salts thus obtained are transformed to pharmaceutical compositions. Further advantages of the use of salts are the better solubility in water, the better wetting properties of the salts, furthermore, that salts can be prepared in higher purity in the most cases than the corresponding bases.

The purpose of our development was the preparation of high purity salts of aripiprazole having advantageous properties for the preparation of pharmaceutical compositions, which can be prepared easily on industrial scale, with environment protecting and reproducible process.

The aim described above is solved according to the present invention.

### SUMMARY OF THE INVENTION

We found surprisingly, that the salts of aripiprazole with dibasic organic acids, camphorsulfonic acid and phosphoric acid according to the formula

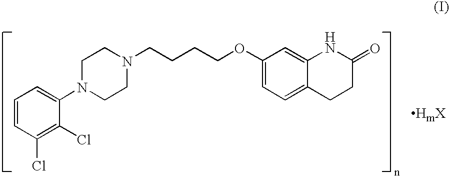

have very advantageous solubility and appropriate hydrophobic properties and their use is very advantageous in the pharmaceutical industry.

### DETAILED DESCRIPTION OF THE INVENTION

Aripiprazole salts with dibasic organic acids, according to general formula (I) wherein X stands for acid radicals of a dibasic organic acid, n stands for 1 or 2, m is 1 or 2 are the objects of present invention.

These compounds have more advantageous properties from the point of view of pharmaceutical formulation than aripiprazole itself or known salts thereof.

Maleic acid, fumaric acid, succinic acid, malic acid, tartaric acid, malonic acid, oxalic acid, or phtalic acid can be used as acids for the preparation of aripiprazole salts. Preferably oxalic acid, tartaric acid or succinic acid can be used.

Further objects of the present invention are the aripiprazole salts of camphorsulfonic acid and phosphoric acid. These compounds have advantageous properties similar to those of salts formed with dibasic organic acids.

Further object of the present invention is a process for the preparation of aripiprazole salts according to general formula (I). Aripiprazole salts according to the present invention are prepared by the reaction of the base of aripiprazole with an appropriate acid in a suitable organic solvent. The precipitated product of the reaction is filtered off and washed with an organic solvent or a mixture of an organic solvent and water if it is necessary.

Suitable solvents are lower alcohols comprising 1-4 carbon atoms, ethers or esters preferably diethyl ether, ethyl acetate, methanol, ethanol, 2-propanol or their mixtures or their mixtures with water can be used for the process.

Acids for the preparation of aripiprazole salts can be used in a molar ratio of 0.5-1.3 based on the molar amount of used aripiprazole base, preferably the acids are used in equimolecular amount.

Further objects of the present invention are the pharmaceutical compositions containing aripiprazole salts according to general formula (I) and known pharmaceutical carriers and auxiliary agents.

The pharmaceutical compositions according to the present invention contain generally 0.1-95 weight %, preferably 1-50 weight %, more preferably 5-30 weight % active ingredient based on the weight of the composition.

Pharmaceutical compositions according to the present invention can be administered orally (e.g. powders, tablets, filmtablets, capsules, microcapsules, solutions, suspensions or emulsions), parenterally (e.g. intravenous, intramuscular, subcutan or intraperitonial injections or infusion compositions) or rectally (e.g. suppositories), transdermally (e.g. patches), or as an implant, or topically (e.g. creams, ointments or patches).

Dosage unit forms according to the present invention are galenical forms e.g. tablets, injections or suppositories which contain an appropriate amount of the active ingredient.

Either solid or liquid pharmaceutical compositions according to the present invention can be prepared by known methods according to the state of the art.

Solid pharmaceutical compositions for oral administration containing the compound of the general formula (I) can contain carriers and fillers (e.g. lactose, glucose, starch, calcium phosphate, microcrystalline cellulose), binding agents (e.g. gelatine, sorbitol, sodium carboximethylstarch, crospovidone), disintegrating agents (e.g. croscaramellose, sodium-carboximethylcellulose, crospovidone), accessories used in processes of tablet preparation (e.g. magnesium stearate, talcum, polyethyleneglicol, silica or silicium dioxide) and tenzides (e.g. sodium laurylsulphate).

Liquid pharmaceutical compositions for oral administration can be solutions, suspensions or emulsions and can contain suspending agents (e.g. gelatine, carboxymethylcellulose), emulsifiers (e.g. sorbitan monooleate), solvents (e.g. water, oils, glycerine, propylene-glycol, ethanol), buffer agents (acetate, phosphate, citrate buffers) and stabilizers (e.g. methyl-4-hydroxy-benzoate).

Liquid dosage forms acceptable for parenteral administration containing the compound of the general formula (I), are aseptic isotonic solutions which can contain besides the solvents other auxiliary agents to control the pH and conserve the composition.

In case of soft pharmaceutical compositions as suppositories containing the compound of the general formula (I) the active ingredient is steadily dispersed in the carrier (e.g. in polyethyleneglycol or cocoa butter).

Further object of present invention is the use of the compound of the general formula (I) for the preparation of a pharmaceutical composition.

Pharmaceutical compositions containing the compound of general formula (I) can be prepared by known methods of the pharmaceutical industry. The active ingredient is admixed with suitable solid or liquid carriers and accessories and converted to a galenical form. The suitable carriers and accessories used in the pharmaceutical industry and the suitable processes for preparing pharmaceutical compositions are described in the literature (Remington's Pharmaceutical Sciences, Edition 18, Mack Publishing Co., Easton, USA, 1990).

Pharmaceutical compositions containing the compound of general formula (I) as active ingredient are packaged as unit dosage forms.

Further object of the present invention is the use of aripiprazole salts of general formula (I) for the preparation of pharmaceutical compositions for the treatment of psychotic diseases, especially for the treatment of schizophrenia or bipolar disorder characterized in that the salts of the compound of the general formula (I) are admixed with pharmaceutically accepted carriers, vehicles and converted to a galenical form.

The present invention concerns the treatment of psychotic diseases, particularly the treatment of schizophrenia and bipolar disorders characterized in that a pharmaceutically effective amount of aripiprazole salt of the general formula (I) is administered to the patient who needs such a treatment.

Further examples show the object of the present invention in a detailed form without limiting the scope of the protection of the present invention to the examples.

### EXAMPLE 1

### Process for the Preparation of Aripiprazole Oxalate (1:1)

Into an apparatus equipped with an intensive stirrer 20 ml of anhydrous ethanol is added then it is heated to boiling. During reflux and intensive stirring 1 g (2.2 mmoles) of aripiprazole is added and dissolved, then 0.3 g (2.2 mmoles) of oxalic acid is added at 70° C. The mixture is heated to its boiling point, then left to cool to room temperature. The precipitated crystalline product is filtered off and washed with ethanol.

Thus, 1.15 g (91.3%) of white crystals are obtained.

Melting point: 202-205° C.

Analysis based on the chemical formula of

C₂₃H₂₇Cl₂N₃O₂OC₂H₂O₄ (538.43):

| | | | | |
|---|---|---|---|---|
| Calculated: | C: 55.77 | H: 5.43 | Cl: 13.17 | N: 7.80 |
| Measured: | C: 56.08 | H: 5.38 | Cl: 12.93 | N: 7.73 |

IR (KBr): 2453, 1683, 1626, 1380, 1170 cm⁻¹.

HNMR (DMSO, i500): 10.00 (bs, 1H), 7.34 (m, 2H), 7.18 (m, 1H), 7.05 (d, J=8.2 Hz, 1H), 6.50 (dd, J₁=2.6 Hz, J₂=8.2 Hz, 1H), 6.45 (d, J=2.4 Hz, 1H), 3.93 (t, J=5.9 Hz, 2H), 3.21 (m, 8H), 3.04 (t, J=7.5 Hz, 2H), 2.79 (˜q, J=7.5 Hz, 2H), 2.42 (t, J=7.6 Hz, 2H), 1.76 (m, 4H), ppm.

CNMR: 170.46, 164.26, 157.92, 150.10, 139.40, 132.89, 128.77, 128.57, 126.25, 125.25, 119.95, 115.80, 107.74, 101.94, 67.05, 55.85, 51.63, 48.66, 30.93, 26.25, 24.18, 20.92, 18.74 ppm.

### EXAMPLE 2

### Process for the Preparation of Aripiprazole Tartarate (1:1)

Into an apparatus equipped with an intensive stirrer 20 ml of anhydrous ethanol is added then it is heated to boiling. During reflux and intensive stirring 1 g (2.2 mmoles) of aripiprazole is added and dissolved, then 0.33 g (2.2 mmoles) of L-(+)-tartaric acid is added at 70° C. The mixture is heated to its boiling point, then left to cool to room temperature. The precipitated crystalline product is filtered off, and washed with ethanol.

Thus, 1.23 g (93.2%) of white crystals are obtained.

Melting point: 190-193° C.

Analysis based on the chemical formula of

C₂₃H₂₇Cl₂N₃O₂O C₄H₆O₆(598.49):

| | | | | |
|---|---|---|---|---|
| Calculated: | C: 54.19 | H: 5.56 | Cl: 11.85 | N: 7.02 |
| Measured: | C: 54.15 | H: 5.50 | Cl: 11.77 | N: 7.02 |

IR (KBr): 3367, 2604, 1673, 1375, 1119 cm⁻¹.

HNMR (DMSO, i500): 9.98 (bs, 1H), 7.31 (m, 2H), 7.15 (m, 1H), 7.04 (d, J=8.2 Hz, 1H), 6.49 (dd, J₁=2.6 Hz, J₂=8.3 Hz, 1H), 6.44 (d, J=2.5 Hz, 1H), 4.23 (s, 2H), 3.93 (t, J=6.3 Hz, 2H), 3.04 (m, 4H), 2.78 (t, J=7.4 Hz, 2H), 2.72 (m, 4H), 2.57 (t, J=7.3 Hz, 2H), 2.41 (t, J=7.5 Hz, 2H), 1.73 (˜qn, J=7.1 Hz, 2H), 1.64 (˜qn, J=6.8 Hz, 2H) ppm.

CNMR: 173.64, 170.46, 158.03, 151.04, 139.37, 132.81, 128.65, 128,56, 126.20, 124.70, 119.79, 115.68, 107.74, 101.90, 72.22, 67.35, 57.03, 52.57, 50.45, 30.94, 26.64, 24.18, 22.34 ppm.

### EXAMPLE 3

### Process for the Preparation of Aripiprazole Hemisuccinate (2:1)

Into an apparatus equipped with an intensive stirrer 20 ml of anhydrous ethanol is added then it is heated to boiling. During reflux and intensive stirring 1 g (2.2 mmoles) of aripiprazole is dissolved, then 0.24 g (2.2 mmoles) of succinic acid is added at 70° C. The mixture is heated to its boiling point, then left to cool to room temperature. The precipitated crystalline product is filtered off, and washed with ethanol.

Thus, 1.01 g (80.8%) of white crystals are obtained.

Melting point: 154-156° C.

Analysis based on the chemical formula of

C₂₃H₂₇Cl₂N₃O₂0½C₄H₆O₄ (507.44):

| | | | | |
|---|---|---|---|---|
| Calculated: | C: 59.18 | H: 5.96 | Cl: 13.97 | N: 8.28 |
| Measured: | C: 59.15 | H: 6.09 | Cl: 14.06 | N: 8.20 |

IR (KBr): 2943, 1689, 1628, 1378, 957 cm⁻¹.

HNMR (CDCl₃, i500): 11.74 (b, 1H), 9.15 (bs, 1H), 7.18 (dd, J₁=2.0 Hz, J₂=8.1 Hz, 1H), 7.15 (˜t, J=7.9 Hz, 1H), 7.02 (d, J=8.3 Hz, 1H), 6.96 (dd, J1=1.8 Hz, J2=7.7 Hz), 6.50 (dd, J₁=2.5 Hz, J₂=8.2 Hz, 1H), 6.39 (d, J=2.4 Hz, 1H), 3.97 (m, 2H), 3.16 (m, 4H), 2.91 (m, 4H), 2.87 (t, J=7.5 Hz, 2H), 2.70 (m, 2H), 2.61 (s, 2H), 2.60 (t, J=7.2 Hz, 2H), 1.80 (m, 4H) ppm.

CNMR: 177.46, 172.43, 158.33, 150.36, 138.12, 134.03, 128.57, 127.53, 127.47, 125.01, 118.72, 115.64, 109.01, 102.11, 67.45, 57.41, 52.52, 50.05, 30.91, 30.89, 26.74, 24.43, 22.15 ppm.

### EXAMPLE 4

### Process for the Preparation of Aripiprazole Camphorsulfonate

Into an apparatus equipped with an intensive stirrer 20 ml of anhydrous ethanol is added then it is heated to boiling. During reflux and intensive stirring 1 g (2.2 mmoles) of aripiprazole is added and dissolved, then 0.55 g (2.2 mmoles) of 1(S)-(+)-camphorsulfonic acid monohydrate is added at 70° C. The mixture is heated to its boiling point, then left to cool to room temperature. The precipitated crystalline product is filtered off, and washed with ethanol.

Thus, 1.33 g (86.4%) of white crystals are obtained.

Melting point: 190-192° C.

Analysis based on the chemical formula of

C₂₃H₂₇Cl₂N₃O₂O C₁₀H₁₆O₄S (680.70)

| | | | | |
|---|---|---|---|---|
| Calculated: | C: 58.23 | H: 6.37 | Cl: 10.42 | N: 6.17 |
| Measured: | C: 58.28 | H: 6.33 | Cl: 10.32 | N: 6.26 |

IR (KBr): 3252, 1739, 1700, 1186, 1030 cm⁻¹.

HNMR (CDCl₃, i500): 10.98 (b, 1H), 8.73 (bs, 1H), 7.23 (dd, J₁=1.5 Hz, J₂=7.9 Hz, 1H), 7.18 (˜t, J=8.0 Hz, 1H), 7.11 (dd, J₁=1.5 Hz, J₂=7.9 Hz, 1H), 7.01 (d, J=8.3 Hz, 1H), 6.53 (d, J=2.3 Hz, 1H), 6.48 (dd, J₁=2.5 Hz, J₂=8.3 Hz, 1H), 4.01 (t, J=6.0 Hz, 2H), 3.77 (m, 2H), 3.58 (m, 2H), 3.38 (m, 2H), 3.33 (d, J=14.5 Hz, 1H), 3.25 (m, 2H), 3.09 (m, 2H), 2.89 (d, J=14.7 Hz, 1H), 2.86 (t, J=7.7 Hz, 2H), 2.70 (m, 1H), 2.58 (t, J=7.5 Hz, 2H), 2.32 (m, 1H), 2.05 (m, 4H, 1.88 (m, 3H), 1.77 (m, 1H), 1.37 (m, 1H), 1.10 (s, 3H), 0.83 (s, 3H) ppm.

CNMR: 217.03, 171.54, 158.10, 149.07, 138.53, 133.99, 128.49, 127.89, 127.57, 126.02, 119.59, 115.98, 108.75, 102.48, 67.17, 58.44, 57.17, 52.38, 48.06, 47.94, 47.60, 42.93, 42.58, 31.04, 26.98, 26.25, 24.57, 20.82, 19.88, 19.79 ppm.

### EXAMPLE 5

### Process for the Preparation of Aripiprazole Phosphate Monohydrate (1:1)

Into an apparatus equipped with an intensive stirrer 20 ml of anhydrous ethanol is added then it is heated to boiling. During reflux and intensive stirring 1 g (2.2 mmoles) of aripiprazole is added and dissolved, then 0.25 g (4.4 mmoles) of 85 weight % phosphoric acid is added at 70° C. The mixture is heated to its boiling point, then left to cool to room temperature. The precipitated crystalline product is filtered off, and washed with ethanol.

Thus, 0.78 g (65.0%) of white crystals are obtained.

Melting point: 189-192° C.

IR (KBr): 2942, 1655, 1592, 1192, 1077, 956 cm⁻¹.

HNMR (DMSO, i500): 10.01 (bs, 1H), 7.32 (m, 2H), 7.16 (m, 1H), 7.04 (d, J=8.3 Hz, 1H), 6.50 (dd, J₁=2.6 Hz, J₂=8.3 Hz, 1H), 6.45 (d, J=2.4 Hz, 1H), 3.93 (t, J=6.1 Hz, 2H), 3.08 (m, 4H), 2.78 (m, 6H), 2.64 (t, J=6.8 Hz, 2H), 2.41 (t, J=7.5 Hz, 2H), 1.73 (m, 2H), 1.68 (m, 2H) ppm.

CNMR: 170.48, 158.01, 150.88, 139.37, 132.82, 128.68, 128.56, 126.21, 124.80, 119.84, 115.70, 107.77, 101.92, 67.33, 56.80, 52.36, 30.94, 26.58, 24.18, 22.04 ppm.

1. Salts of 7-{444-(2,3-dichlorophenyl)-1-piperazinyllbutoxy13,4-dihydro-2(1R)-quinolinon (arpiprazol) formed with dibasic organic acids of the general formula 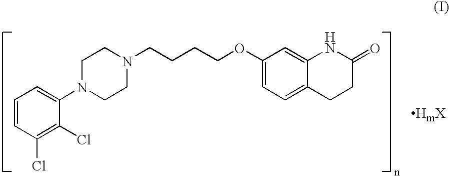
wherein
X stands for acid radicals of a dibasic organic acid,
n stands for 1 or 2, and
m is 1 or 2.

2. Salts of aripiprazole formed with dibasic organic acids according to claim 1 wherein X stands for an acid radical of oxalic acid, tartaric acid or succinic acid.

3. Aripiprazole oxalate (1:1) defined in claim 2.

4. Aripiprazole tartarate (LI) defined in claim 2.

5. Aripiprazole succinate (2:1) defined in claim 2.

6. Aripiprazole camphorsulfonate.

7. Aripiprazole phosphate monohydrate (1:1).

8. A process for the preparation of salts of aripiprazole of the general formula (1) according to claim 1 wherein aripiprazole base is reacted with an appropriate organic acid in a suitable organic solvent, then the precipitated salts are isolated.

9. The process according to claim 8 wherein the amount of the organic acid is in a mole ratio of 0.5-3 moles based on the molar amount of aripiprazole base.

10. The process according to claim 8 wherein alcohols, ethers or esters containing 1-4 carbon atoms or mixtures thereof are used as solvent.

11. Pharmaceutical compositions containing aripiprazole salts according to claim 1 as active pharmaceutical ingredient and pharmaceutically acceptable carriers.

12. A process for preparation of pharmaceutical composition according to claim 11 wherein a compound of the general formula (1) of claim 1 is admixed with at least one pharmaceutically acceptable carrier and if necessary with further pharmaceutically acceptable auxiliary agents and converted to a galenical form.

13. A use of aripiprazole salts of the general formula (I) according to claim 1 for the treatment of psychotic diseases of the central nervous system.

14. A method of treatment or prevention of psychotic diseases of the central nervous system wherein at least one aripiprazole salt of the general formula (I) according to claim 1 is administered in a pharmaceutically efficient amount to the patient who needs such treatment.
